(19) United States
(12) Patent Application Publication
(10) Pub. No.: US 2026/0232383 A1
(43) Pub. Date: Aug. 13, 2026

(54) SYSTEM AND METHOD FOR VISUAL IMAGE GUIDANCE DURING A MEDICAL PROCEDURE

(71) Applicants: BARCO N.V., Kortrijk (BE); KATHOLIEKE UNIVERSITEIT LEUVEN, Leuven (BE)

(72) Inventors: Viktor VÖRÖS, Kessel-Lo (BE); Emmanuel VANDER POORTEN, Mechelen (BE)

(73) Assignees: BARCO N.V., Kortrijk (BE); KATHOLIEKE UNIVERSITEIT LEUVEN, Leuven (BE)

(21) Appl. No.: 19/150,642

(22) PCT Filed: Jan. 24, 2024
(86) PCT No.: PCT/EP2024/051674

§ 371 Date: Jul. 24, 2025

(30) Foreign Application Priority Data
Jan. 24, 2023 (EP) 23153173.2

(51) Int. Cl. A61B 34/20; A61B 90/00

(52) U.S. Cl. CPC A61B 34/20; A61B 90/361; A61B 90/37; A61B 2034/2051; A61B 2034/2061; A61B 2090/365; A61B 2090/367

(57) ABSTRACT

A system for providing a visualization during a medical procedure or a simulated medical procedure. The system including means for receiving imaging data; means for receiving tracking data of one or more instruments; and means for receiving model data, and a processing means adapted for co-registering tracking data and model data. The tracking data including tracking points that are not colinear; and/or one or more tracking points and a roll, pitch and yaw angle or a set of Euler angles or a set of quaternions or an angle-axis representation or a rotation matrix or a homogeneous transformation matrix; and/or a point cloud representation, for at least one instrument. The tracking data being used to determine a position and/or shape and/or orientation of the instrument; and the visualization includes a representation of the shape and/or the position and/or the orientation of the instrument.

### FIELD OF THE DISCLOSURE

The present disclosure relates to the field of visual image guidance during medical procedures or simulated medical procedures.

### BACKGROUND

Advancements in medical technologies have enabled the development and regular use of minimally invasive procedures, such as for instance laparoscopy or coronary catheterization. These minimally invasive procedures reduce operative trauma, wound healing time, associated pain and risk of infection and other complications. However, it remains a challenge to provide proper visual guidance to the physician executing these procedures. Typically, physicians rely on intraoperative endoscopy and/or fluoroscopy images, potentially combined with pre-operative imaging techniques such as CT-scan or MRI, as well as their experience and knowledge of human anatomy. This way of working is not without its drawbacks: endoscopes have a limited field of view, fluoroscopy and pre-operative imaging techniques require expensive equipment and expose the patient to harmful radiation. In addition, the overwhelming majority of these techniques provide 2D images. The burden of properly estimating the position and orientation, often referred to as pose, of a 3D instrument within the cavity of the patient's body thus still rests on the physician.

Document US20200188028A1 discloses a system and method for providing an augmented reality visualization during a medical procedure on a patient. According to US20200188028A1, a three-dimensional (3D) model of an anatomic part of the patient is presented on a 3D display in alignment with two-dimensional (2D) live imaging data obtained during the procedure. In addition, some embodiments of US20200188028A1 utilize 3D tracking data to localize instruments used in the procedure in real time. The visualization of the position of the instruments can be presented on the 3D display in alignment with the 3D model and/or the 2D live imaging data to guide a clinician in performing the medical procedure.

This solution for visual image guidance during a medical procedure does not consider that the instrument can extend in three dimensions; it tracks one point of the physical instrument and assumes that the corresponding part of the instrument always fits inside the patient's cavity. However, the tip of an endoscope for example can be bent in order to obtain a different camera view angle. If the clinician would rotate such a bent tool around its own axis, inside a patient cavity, he/she could harm the patient's internal and/or wall tissue.

Therefore, there is still a need for an alternative and improved system and method for visual image guidance during a medical procedure.

### SUMMARY

The present disclosure aims to remedy the above-mentioned and other disadvantages.

According to a first aspect of the disclosure, a system for providing a visualization during a medical procedure on a patient or a simulated medical procedure on a patient model is disclosed, the system comprising:
a. means for receiving imaging data of a first region of the patient's body or the patient model;
b. means for receiving tracking data of one or more instruments that are inserted in a second region of the patient's body or the patient model;
c. means for receiving pose data of the patient's body or the patient model, the pose data belonging to a third region;
d. processing means for co-registering the imaging data, tracking data and pose data;
e. a display for simultaneous visualization of two or more of the first, second and third regions, and of the co-registered data belonging to the simultaneously visualized regions;
the system being characterized in that
f. the tracking data comprises three or more tracking points, wherein the three tracking points are not colinear; and/or one or more tracking points and a roll, pitch and yaw angle or a set of Euler angles or a set of quaternions or an angle-axis representation or a rotation matrix or a homogeneous transformation matrix; and/or a point cloud representation, for at least one of the one or more instruments;
g. the tracking data is used to determine a position and/or shape and/or orientation of at least one of the one or more instruments; and
h. the visualization comprises a representation of the shape and/or the position and/or the orientation of at least one of the one or more instruments.

According to preferred embodiments, the system further comprises means for receiving model data of a fourth region of the patient's body or the patient model, wherein the processing means are adapted for co-registering the imaging data, tracking data, pose data and model data, wherein the display is adapted for simultaneous visualization of two or more of the first, second, third and fourth regions, and of the co-registered data belonging to the simultaneously visualized regions.

According to preferred embodiments:
a. the model data and tracking data comprise 3D data;
b. the display is a 3D display; and
c. a representation of the model data and the representation of the shape and/or position and/or orientation of the one or more instruments are 3D representations.

According to preferred embodiments, the imaging data is 2D data that is processed to allow for visualization on the 3D display.

According to preferred embodiments, there is at least a partial overlap between two or more regions and wherein the visualization comprises an overlay of the representation of the data originating from the two or more regions.

According to preferred embodiments, the overlay is presented aligned on corresponding data or wherein the overlay can be positioned and repositioned during the procedure such as to offer a desired view for a user.

According to preferred embodiments, the system latency is equal to or smaller than 50 ms.

According to preferred embodiments, the co-registration of the model data, imaging data, tracking data and orientation data occurs in a continuous or intermittent manner during the procedure.

According to preferred embodiments, the model data is generated prior to the procedure.

According to preferred embodiments, imaging data is generated during the surgical procedure.

According to preferred embodiments, the imaging data is generated in an intermittent manner.

According to preferred embodiments, one or more sensors for generating the tracking data are embedded in or fixedly attached to at least one of the one or more instruments.

According to preferred embodiments, at least one of the one or more sensors comprises an optical fiber and/or wherein at least one of the one or more sensors comprises one or more electromagnetic trackers and/or wherein at least one of the one or more sensors is a distributed sensor that conforms to the shape of the instrument.

According to preferred embodiments, one or more cameras or LIDAR systems have an unobstructed view on at least one of the one or more instruments to derive the tracking data based on image processing techniques applied on the camera or LIDAR images.

According to a second aspect of the disclosure, a method for providing a visualization during a medical procedure on a patient or simulated medical procedure on a patient model is disclosed, the method comprising the steps of:
a. receiving image data of a first region of the patient's body or the patient model;
b. receiving tracking data of one or more instruments that are inserted in a second region of the patient's body or the patient model;
c. receiving pose data of the patient's body or the patient model, belonging to a third region;
d. co-registering the imaging data, tracking data and pose data;
e. simultaneously visualizing of two or more of the first, second and third regions, and of the co-registered data belonging to the simultaneously visualized regions;
the method being characterized in that
f. the tracking data comprises three or more tracking points, wherein the three tracking points are not colinear; and/or one or more tracking points and a roll, pitch and yaw angle or a set of Euler angles or a set of quaternions or an angle-axis representation or a rotation matrix or a homogeneous transformation matrix; and/or a point cloud representation, for at least one of the one or more instruments;
g. the tracking data is used to determine a position and/or shape and/or orientation of at least one of the one or more instruments; and
h. the visualization comprises a representation of the shape and/or the position and/or the orientation of at least one of the one or more instruments.

In some embodiments of the method, the method further comprises the steps of:
a. receiving model data of a fourth region of the patient's body or the patient model;
b. co-registering the imaging data, tracking data and pose data;
simultaneously visualizing two or more of the first, second, third and fourth regions, and of the co-registered data belonging to the simultaneously visualized regions.

According to a third aspect of the disclosure, a system is disclosed for providing a visualization during a medical procedure on a patient or a simulated medical procedure on a patient model, the system comprising:
a. means for receiving imaging data of a first region of the patient's body or the patient model;
b. means for receiving tracking data of one or more instruments that are inserted in a second region of the patient's body or the patient model;
characterized in that
c. the system comprises means for receiving model data of a fourth region of the patient's body or the patient model, and a processing means adapted for co-registering tracking data and model data;
d. the system comprises a display for simultaneous visualization of the co-registered data and the imaging data;
e. the tracking data comprises three or more tracking points, wherein the three tracking points are not colinear; and/or one or more tracking points and a roll, pitch and yaw angle or a set of Euler angles or a set of quaternions or an angle-axis representation or a rotation matrix or a homogeneous transformation matrix; and/or a point cloud representation, for at least one of the one or more instruments;
f. the tracking data is used to determine a position and/or shape and/or orientation of at least one of the one or more instruments; and
g. the visualization comprises a representation of the shape and/or the position and/or the orientation of at least one of the one or more instruments.

According to preferred embodiments, the system further comprises means for receiving pose data of the patient's body or the patient model, the pose data belonging to a third region, wherein the processing means are adapted for further co-registering pose data, wherein the display is adapted for further visualization of the pose data.

According to preferred embodiments, the model data and tracking data comprise 3D data;
a. a shape of the instruments is determined according to the tracking data comprising time-related data;
b. the display is a 3D display; and
c. a representation of the model data and the representation of the shape and/or position and/or orientation of the one or more instruments are 3D representations.

According to preferred embodiments, the imaging data is 2D data that is processed to allow for visualization on the 3D display.

According to preferred embodiments, there is at least a partial overlap between two or more regions. The visualization comprises an overlay of the representation of the data originating from the two or more regions.

According to preferred embodiments, the overlay is presented aligned on corresponding data or wherein the overlay can be positioned and repositioned during the procedure such as to offer a desired view for a user.

According to preferred embodiments, the system latency is equal to or smaller than 50 ms.

According to preferred embodiments, the co-registration of the model data, imaging data, tracking data and orientation data occurs in a continuous or intermittent manner during the procedure.

According to preferred embodiments, the model data is generated prior to the procedure.

According to preferred embodiments, the imaging data is generated during the surgical procedure.

According to preferred embodiments, the imaging data is generated in an intermittent manner.

According to preferred embodiments, one or more sensors for generating the tracking data are embedded in or fixedly attached to at least one of the one or more instruments.

According to preferred embodiments, at least one of the one or more sensors comprises an optical fiber and/or wherein at least one of the one or more sensors comprises one or more electromagnetic trackers and/or wherein at least one of the one or more sensors is a distributed sensor that conforms to the shape of the instrument.

According to preferred embodiments, one or more cameras or LIDAR systems have an unobstructed view on at least one of the one or more instruments to derive the tracking data based on image processing techniques applied on the camera or LIDAR images.

According to a fourth aspect of the disclosure, a method is disclosed for providing a visualization during a medical procedure on a patient or simulated medical procedure on a patient model, the method comprising the steps of:
a. receiving image data of a first region of the patient's body or the patient model;
b. receiving tracking data of one or more instruments that are inserted in a second region of the patient's body or the patient model;
   the method being characterized in that it comprises:
c. receiving model data of the patient's body or the patient model;
d. co-registering the tracking data and model data;
e. simultaneously visualizing imaging data and of the co-registered data;
and in that
f. the tracking data comprises three or more tracking points, wherein the three tracking points are not colinear; and/or one or more tracking points and a roll, pitch and yaw angle or a set of Euler angles or a set of quaternions or an angle-axis representation or a rotation matrix or a homogeneous transformation matrix; and/or a point cloud representation, for at least one of the one or more instruments;
and in that
g. the tracking data is used to determine a position and/or shape and/or orientation of at least one of the one or more instruments; and
h. the visualization comprises a representation of the shape and/or the position and/or the orientation of at least one of the one or more instruments.

According to preferred embodiments, the method further comprising receiving pose data of the patient's body or the patient model, the pose data belonging to a third region, further co-registering pose data, further visualizing of the pose data.

According to preferred embodiments,
a. the model data and tracking data comprise 3D data;
b. the display is a 3D display; and
c. a representation of the model data and the representation of the shape and/or position and/or orientation of the one or more instruments are 3D representations.

According to preferred embodiments, the imaging data is 2D data that is processed to allow for visualization on the 3D display.

According to preferred embodiments, there is at least a partial overlap between two or more regions and wherein the visualization comprises an overlay of the representation of the data originating from the two or more regions.

According to preferred embodiments, the overlay is presented aligned on corresponding data or wherein the overlay can be positioned and repositioned during the procedure such as to offer a desired view for a user.

According to preferred embodiments, the system latency is equal to or smaller than 50 ms.

According to preferred embodiments, the co-registration of the model data, imaging data, tracking data and orientation data occurs in a continuous or intermittent manner during the procedure.

According to preferred embodiments, the model data is generated prior to the procedure.

According to preferred embodiments, the imaging data is generated during the surgical procedure.

According to preferred embodiments, the imaging data is generated in an intermittent manner.

According to preferred embodiments, one or more sensors for generating the tracking data are embedded in or fixedly attached to at least one of the one or more instruments.

According to preferred embodiments, at least one of the one or more sensors comprises an optical fiber and/or wherein at least one of the one or more sensors comprises one or more electromagnetic trackers and/or wherein at least one of the one or more sensors is a distributed sensor that conforms to the shape of the instrument.

According to preferred embodiments, one or more cameras or LIDAR systems have an unobstructed view on at least one of the one or more instruments to derive the tracking data based on image processing techniques applied on the camera or LIDAR images.

According to a further aspect of the present disclosure, a method is disclosed for providing a visualization during of a target region on a patient during a medical procedure, comprising the steps of:
receiving imaging data and model data of the target region;
receiving tracking data of a flexible instrument;
generating a 2D image according to the image data;
   wherein the method further comprises:
co-registering the model data and the tracking data to generate a 3D image;
simultaneously visualizing the 2D image and the 3D image on a 3D display;
   and wherein the tracking data is used to determine a position and/or shape and/or orientation of at least one of the one or more instruments.

According to preferred embodiments, the method further comprises:
receiving pose data of the patient, wherein the pose data is used to determine position and orientation of the patient;
co-registering the pose data to the model data and the tracking data, wherein the 3D image is displayed on the 3D display in a parallel orientation of the patient.

According to preferred embodiments, the 2D image and the 3D image are displayed side-by-side or overlayed over each other on the 3D display.

According to preferred embodiments, the co-registration of the model data, imaging data, tracking data occurs in a continuous or intermittent manner during the procedure.

According to preferred embodiments, the co-registration of the model data, pose data, tracking data occurs in a continuous or intermittent manner during the procedure.

According to preferred embodiments, the latency of displaying is equal to or smaller than 50 ms.

The following further explains different features of the first and third aspect of the present disclosure, and of the second and fourth aspect, mutatis mutandis.

In the context of the present disclosure, “imaging data” refers to data that represents one or more visual images of one or more parts of the interior of the patient's body or the patient model. Alternatively, or in addition, imaging data may represent visual images of one or more parts of the instruments or of a physician operating the instruments. For instance, the imaging data may comprise a visual image of an interior region of the patient's body, together with a part of an instrument inserted into said region and with the physician's hand holding the instrument. Alternatively, or in addition, imaging data may refer to non-visual data of the patient's body or patient model that is recorded, such as for instance heart rate, blood pressure or oxygen saturation levels.

In the context of the present disclosure, “tracking data” refers to data that can be used to retrieve a spatial position and/or orientation of the one or more instruments. Alternatively, or in addition, tracking data may also comprise time-related data such as time stamps, an indication of chronology, propagation speeds etcetera.

In the context of the present disclosure, “pose data” refers to the ensemble of position and orientation. In case of a human patient, the pose data refers amongst other to the position and orientation of the head, trunk and limbs. The pose data may concern the entirety of the patient's body or only a part of it. In case of an instrument, the pose data can refer amongst others to the position and the orientation of a point of the instrument, e.g. the instrument tip, or the instrument handle, or any other convenient and representative point on the instrument. When the one or more instruments are steerable instruments, the combination of the pose data offers a representation of the shape of the instrument. The pose data at different points may alter relative to each other. By tracking individual position and orientation points and fitting splines or other representations, the shape of the steerable instrument may be computed and visualized accordingly.

In the context of the present disclosure, “co-registration” refers to the process of aligning data that originates in different reference frames. This co-registration may require translating, rotating or scaling data, both in the spatial and the temporal domain.

The tracking data of at least one of the one or more instruments comprises three or more tracking points, wherein the three tracking points are not colinear. Knowledge of the position of three non-colinear tracking points allows to determine the position and orientation of the instrument in an unambiguous manner, when the tracking points have a fixed geometric relationship to the instrument. Preferably, the instrument comprises tracking points that are distributed over its geometrical structure such that the shape of the instrument can be estimated through knowledge of the tracking points.

Alternatively, the tracking data of at least one of the one or more instruments comprises one or more tracking points, and/or representation of the corresponding orientation(s) either by means of the corresponding roll, pitch and yaw angle, set of Euler angles, set of quaternions, angle-axis representation, rotation matrix, homogeneous transformation matrix or any other representation of orientation. Knowledge of these parameters also allows for an unambiguous determination of the position and orientation of the instrument, but only when the tracking point has a fixed or known geometric relationship to the instrument. For instruments that are deformable and adjustable in shape, the shape of the instrument can be estimated provided there are sufficient tracking points distributed over the instrument, or a point cloud representation of the instrument's shape, or a mechanical or other type of relation that allows estimating the entire instrument's shape based on input parameters such as actuation force, pressure or other input parameters.

It is an advantage of the system that not only the shape, but also the position and orientation of the instruments can be visualized. If the shape, position and/or orientation of the instruments are visualized on the display, the physician performing the procedure does not need to rely solely on knowledge, experience and feel to estimate whether the instruments are properly positioned and fit inside the anatomical structure where they are inserted. This may allow for faster, safer and more effective medical procedures.

In some embodiments of the system of the first aspect, the system further comprises means for receiving model data of a fourth region of the patient's body or the patient model. In addition, the processing means are adapted for co-registering the imaging data, tracking data, pose data and model data, and the display is adapted for simultaneous visualization of two or more of the first, second, third and fourth regions, and of the co-registered data belonging to the simultaneously visualized regions.

In the context of the present disclosure, “model data” refers to data that offers a visual representation of one or more parts of the interior of the patient's body or the patient model. When compared to “imaging data”, model data may offer higher spatial resolution. Model data is often based on various sources of imaging data that have been processed by a computer to generate a single, more complex, data set. In addition, model data may comprise visual indications of properties that are not visually discernible in real life.

In some embodiments of the system of the third aspect, the system further comprises means for receiving pose data of the patient's body or the patient model, the pose data belonging to a third region, wherein the processing means are adapted for further co-registering pose data, wherein the display is adapted for further visualization of the pose data.

In some embodiments of the system comprising model data, the model data and tracking data comprise 3D data, the display is a 3D display and a representation of the model data and/or the representation of the shape and/or position and/or orientation of the one or more instruments are 3D representations. Preferably, the shape of the instruments is determined according to the tracking data comprising time-related data.

Anatomical structures and instruments employed during medical procedures inherently have a 3D shape. When these 3D shapes are projected onto a 2D plane, as happens during visualization on a traditional 2D display, a lot of information is lost. Often, it is necessary to present a 2D view from multiple angles in order to properly assess a situation. Even then, the geometric relations between the different objects shown on the 2D display may not be directly clear and unambiguous. The usage of a 3D display, in conjunction with 3D model and tracking data, solves these problems.

In some embodiments of the system employing a 3D display, the imaging data is 2D data that is processed to allow for visualization on the 3D display.

Typically, live imaging data obtained during a medical procedure is of a 2D nature. Displaying the 2D imaging data as 3D data could introduce visualization artifacts and/or give the false impression that additional geometric information is contained in the data. To visualize data on a 3D display, such as an autostereoscopic display or XR glasses or any other type of 3D visualization system, two virtual cameras are placed in the scene that is to be rendered. Each of these cameras will provide the image for a separate eye of the user. By making the positions and orientations of these cameras coincide, both eyes of the user are presented with the same images. The resulting image that is visualized on the display is thus perceived as a 2D image. This technique allows for the proper visualization of a 2D image on a 3D display. It also allows for the conversion of a native 3D image to a 2D image.

In some embodiments of the system, there is at least a partial overlap between two or more of the identified regions and the visualization comprises an overlay of the visualization of the data originating from the overlapping regions. For instance, the amount of spatial overlap between the regions can be 0%, 20%, 50% or 100% of the surface area of the smallest of the regions. In case of overlap between three or more regions, the amount of overlap is not necessarily identical between every pair of regions among the three or more regions.

For instance, there may be a partial spatial overlap between the second region and the fourth region and the visualization comprises an overlay of the representation of the shape and/or the position and/or the orientation of at least one of the one or more instruments on a representation of the model data.

Preferably, there is a significant spatial overlap between the second region-comprising the inserted instruments- and the fourth region-comprising the model data. Preferably, there is also a significant spatial overlap between both regions and the target region of the medical procedure or the simulated medical procedure. Preferably, the overlay comprises a representation of the shape and position and orientation of all of the instruments inserted into the patient or the patient model, and a representation of the model data. The resulting visualization allows a physician to see in real time and 3D where and how the instruments are positioned within the model representation of the patient's body or the patient model. If the model data is acquired from actual measurements on the patient's body or the patient model and is sufficiently accurate, the resulting visualization allows a physician to interpret in real time and 3D where and how the instruments are positioned within the patient's body or the patient model.

For instance, there may be a partial spatial overlap between the first region and the second region and the visualization comprises an overlay of the representation of the shape and/or the position and/or the orientation of at least one of the one or more instruments on a representation of the imaging data.

Preferably, there is a significant spatial overlap between the second region-comprising the inserted instruments- and the first region-comprising the imaging data. Preferably, there is also a significant spatial overlap between both regions and the target region of the medical procedure or the simulated medical procedure. Preferably, the overlay comprises a representation of the shape and position and orientation of all of the instruments inserted into the patient or the patient model, and a representation of the imaging data. The resulting visualization allows a physician to see in real time where and how the instruments are positioned within the patient's body or the patient model.

In some embodiments of the system wherein the visualization comprises an overlay, the overlay is presented aligned on corresponding data or the overlay can be positioned and repositioned during the procedure such as to offer a desired view for a user.

Because of the co-registration of the different data streams, the transformations between the different data streams are known. It is thus possible to present the visualizations of two or more different data streams simultaneously where the visualization of data points that correspond to the same physical features are aligned. This allows for the direct and unambiguous verification of where and how the instruments are positioned within the patient's body or the patient model and of whether they risk to damage any of the surrounding tissues. Alternatively, both visualizations may be positioned independently of one another, without aligning the corresponding data points, in order to obtain a clearer view of the representation of one or more of the data streams.

The skilled person understands that it is possible to display more than two different data streams simultaneously in overlay. For instance, both the model data and the tracking data may be overlaid on the imaging data.

In some embodiments of the system, the latency of the system is equal to or smaller than 50 ms. Preferably, the latency of the system is inferior to 40 ms, more preferably inferior to 30 ms, most preferably inferior to 20 ms. The skilled person understands that the system latency may comprise the time needed for raw data generation and acquisition, eventual pre-processing of the raw data, the time required to estimate or calculate the transformations between the respective reference frames of the different data streams, the time required to co-register the different data streams, based on the obtained transformations and the time required for the rendering and visualization pipeline.

In order to be useful for providing real-time visual guidance during a medical procedure, it is imperative that the latency of the system is sufficiently small. Given the small size of some anatomical structures, a significant latency between the actions of the physician, and hence the receipt of the raw data, and the visualization of said data could lead to the physician advancing an instrument too far before feedback is received, thereby inducing an unintentional collision of the instrument with the tissue of the anatomical structure. By keeping the maximum total latency of the system in the same order of magnitude as the time the human brain needs to acquire and process an image, this risk is mitigated. In addition, perceptible system latency can be fatiguing and/or frustrating for the physician.

In some embodiments of the system, the co-registration of the model data, imaging data, tracking data and orientation data occurs in a continuous or intermittent manner during the procedure.

The time required for the estimation or calculation of the transformations between the respective reference frames of the different data streams makes a significant contribution to the total system latency. Therefore, the total system latency and/or the required computational power can be significantly reduced if the co-registration of the data streams is not updated for every frame that is displayed, but rather in an intermittent fashion. The intermittent co-registration process may be triggered in a variety of ways. It may for instance occur after a set number of frames have been visualized, or when prompted by a user of the system, or when a certain amount of movement of one of the reference frames has been detected.

In some embodiments of the system, the model data is generated prior to the procedure.

The generation of model data generally relies on a large amount of imaging data, wherein this imaging data is heavily processed. Therefore, the generation of model data is a computationally intensive task. The total system latency and/or the required computational power can thus be significantly reduced if model data is generated prior to the medical or simulated medical procedure.

In some embodiments of the system, the model data is generated using computed tomography, MRI or any other suitable modality.

In some embodiments of the system, the imaging data is generated during the surgical procedure.

In some embodiments of the system, the imaging data is generated using fluoroscopy or intraoperative MRI (iMRI) or any other real-time imaging method.

In some embodiments of the system, the imaging data is generated in an intermittent manner. In this context, generating imaging data “in an intermittent manner” is to be understood as opposed to continuously generating imaging data or generating imaging data per frame that is to be visualized.

When operating in an intermittent manner, the generation of imaging data can for instance be controlled by an elapsed time interval, a displayed number of frames, a predetermined amount of movement of one of the instruments, a predetermined amount of variation between successively acquired data streams, a predetermined amount of variation between successive images or an action of the physician performing the procedure. The skilled person is capable of understanding the circumstances at play and of deriving a suitable criterion for the determination of the interval for the generation of imaging data.

There are multiple reasons to generate imaging data in an intermittent manner instead of continuously or on a per frame basis. First of all, typical imaging techniques such as fluoroscopy expose the patient to a non-negligible amount of radiation. By only generating imaging techniques in an intermittent manner, the total radiation exposure of the patient can be greatly reduced. Secondly, the preprocessing of the raw imaging data may be a computationally intensive task, which might negatively impact the total system latency. The total system latency and/or the required computational power can thus be significantly reduced if imaging data is generated in an intermittent fashion. In third place, typical imaging techniques require a finite exposure time. This exposure time may be longer than the total system latency that is desired.

In some embodiments of the system, one or more sensors for generating the tracking data are embedded in or fixedly attached to at least one of the one or more instruments. These sensors may comprise accelerometers, capacitive sensors, inductive sensors, optical sensors, LIDAR or any other kind of sensor that is capable of generating data that allows for the estimation of a position and/or orientation and/or shape.

This embedding or fixedly attaching of sensors ensures that the sensors do not separate from the instruments inside the patient's body. Furthermore, it ensures that the position of the sensors on the instrument is fixed, both in space and in time. Alternatively, the sensors may be attached to the instruments in a movable manner, provided the geometric relationship between the sensor and the instrument is known or can be determined. Consequently, periodic recalibration of the tracking points generated by the sensors with respect to the instrument's shape or orientation is not required.

Preferably, the sensors are embedded in the instruments. This permits the instruments to have a smooth outer surface which is not abrasive towards the tissues inside the patient's body. Furthermore, this ensures that the surface of the instrument does not need to display any substantial relief, gaps or crevices, which can become a breeding ground for pathogenic organisms.

In some embodiments of the system, at least one of the one or more sensors comprises an optical fiber and/or at least one of the one or more sensors comprises one or more electromagnetic trackers and/or at least one of the one or more sensors is a distributed sensor that conforms to the shape of the instrument.

Optical fibers are ideally suited for implementation in medical instruments. They are small in diameter, lightweight, flexible, chemically inert and do not emit electromagnetic noise. Furthermore, they offer a high bandwidth for data transfer and can be used to introduce a light source inside of the patient's body. When inscribed with multiple Fiber Bragg Gratings (FBG) s, distributed along the length of the fiber, the local strain on the fiber can be measured which allows for a reconstruction of the fiber's shape. Based on the estimated shape of the fiber, the position, orientation and shape of the instrument can be determined.

Another option is to use Electromagnetic Trackers (EMTs) embedded in the instrument. A thorough explanation of the combined usage of distributed FBG sensors and EMTs for shape reconstruction of a catheter can be found in:
Ha et al., Robust Catheter Tracking by Fusing Electromagnetic Tracking, Fiber Bragg Grating and Sparse Fluoroscopic Images, IEEE Sensors Journal, Vol. 21, Issue 20, Pages 23422-23434, DOI: 10.1109/JSEN.2021.3107036.

In some embodiments of the system, one or more cameras or LIDAR systems have an unobstructed view on at least one of the one or more instruments to derive the tracking data based on image processing techniques applied on the camera or LIDAR images.

In some procedures and/or for some instruments, it might be possible to position one or more cameras or LIDAR systems such that they retain an unobstructed view of the instrument, even when said instrument is inserted into the patient's body or the patient model. It is an advantage of this technique that it requires no additional sensors inside or on the instruments and can thus be employed with standard instruments that are in widespread use.

### BRIEF DESCRIPTION OF THE DRAWINGS

FIG. 1 schematically illustrates an embodiment of the system according to the present disclosure.

FIGS. 2(a) and 2(b) schematically illustrate a simulated medical procedure.

FIG. 3(a) shows a patient model for use in simulated cardiac catheterization.

FIGS. 3(b)-(f) show various manners to visualize a simulated medical procedure on the patient model of FIG. 3(a).

### DETAILED DESCRIPTION

The present disclosure will be described in terms of specific embodiments, which are illustrative of the disclosure and which are not to be construed as limiting. It will be appreciated that the present disclosure is not limited by what has been particularly shown and/or described and that alternatives or modified embodiments could be developed in the light of the overall teaching of this disclosure. The drawings described are only schematic and are non-limiting.

Reference throughout this description to “one embodiment” or “an embodiment” means that a particular feature, structure or characteristic described in connection with the embodiments is included in one or more embodiment of the present disclosure. Thus, appearances of the phrases “in one embodiment” or “in an embodiment” in various places throughout this specification are not necessarily all referring to the same embodiment, but may do so. Furthermore, the particular features, structures or characteristics may be combined in any suitable manner, as would be apparent to one of ordinary skill in the art from this disclosure, in one or more embodiments.

Specific features, structures or characteristics are indicated in the figures by reference numerals. In order not to overload the figures, not every feature is indicated in every figure. Conversely, in order not to overload the text, not every feature indicated in a figure is also discussed in the context of this particular figure.

Finally, the use of ordinal numbers such as “first”, “second” and the like throughout this disclosure in no way implies a hierarchical relationship—not in terms of importance, position or time—between the features with which they are used, unless explicitly stated to the contrary. These ordinal numbers serve only to differentiate between different but similar features, properties, or structures.

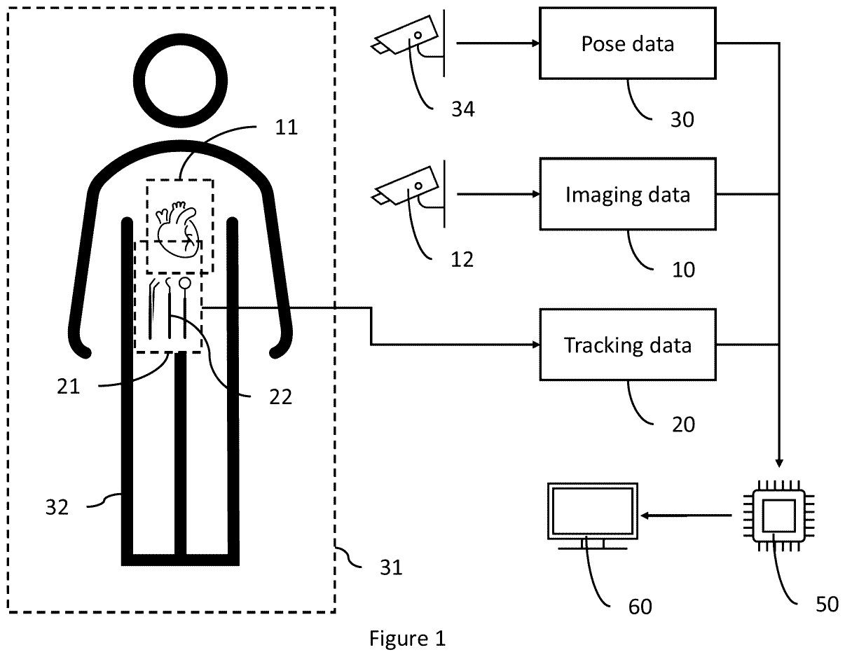

FIG. 1 schematically illustrates an embodiment of the system according to the present disclosure. In the embodiment of FIG. 1, a patient undergoes a medical procedure.

A first imager 12 provides imaging data 10 of a first region 11 of the patient's body 32. The first imager 12 can be for instance an ultrasound, a fluoroscope or an intraoperative magnetic resonance imaging (iMRI) apparatus. Preferably, the first region 11 comprises the target region of the medical procedure. The first imager 12 may be any transducer used during medical procedure to convert from one type of energy signal to an image signal to produce the imaging data 10.

A second imager 34 provides pose data 30 of the patient's body 32, belonging to a third region 31. The second imager can be for instance a camera or, more preferably, two cameras that are arranged to obtain a stereoscopic view of the patient's body 32. Preferably, the pose data 30 comprises at least data on the position and orientation of the body part that comprises the target region of the medical procedure. Preferably, the pose data 30 comprises data on the position and orientation of the patient's trunk and head.

One or more instruments 22 are inserted in a second region 21 of the patient's body 32. The instruments 22 can comprise any kind of surgical or non-surgical tool, such as for instance catheters, cannulas, needles, endoscopes, cutters, graspers, clamps, dilators and the like. The instruments 22 may be any type of flexible instruments that are inserted into the patient's body and tracked during medical procedure. In some embodiments, the instruments 22 may have non-linear shape or direction during medical procedure. Preferably, the second region 21 coincides at least partially with the first region 11. Preferably, the second region 22 comprises the target region of the medical procedure.

At least one of the instruments 22 is equipped with means to generate tracking data 20. The tracking data 20 of a single instrument comprises the positions of at least three non-colinear tracking points. The knowledge of the position of three non-colinear points allows the unambiguous reconstruction of the position and orientation of the instrument. In addition, the three non-linear points may allow for the shape of the instrument to be reconstructed. In case the instrument is flexible or comprises articulations, more tracking points may be required to reconstruct position, orientation, or shape of the instrument. Alternatively, when the shape of the instrument is known, the tracking data 20 of a single instrument may comprise the position of one or more tracking points and the values of a roll, a pitch and a yaw angle. Again, in case the instrument is flexible or comprises articulations, more tracking points and more angles may be required to reconstruct position, orientation or shape of the instrument. Specific techniques for generating tracking data will be discussed further in this disclosure.

The imaging data 10, tracking data 20, and pose data 30 are received by a processor 50. Each of the data streams corresponding to the imaging data 10, tracking data 20, and pose data 30 belongs to a reference frame. The reference frame is related to the position, orientation and properties of the device that generated the data stream. Important differences may exist between the different reference frames. For instance, the imaging data 10 and tracking data 20 may be translated or rotated with respect to one another or may differ in scale. In addition, data streams may be sampled continuously or intermittently acquired at different frequencies. In addition, continuously or intermittently acquired data streams may be sampled at different frequencies. The acquisition of data streams or sampling can be both continuous or intermittent. For example, fluoroscopy is not always continuous, thus the image can be static for a certain amount of time (e.g. seconds or minutes). And for instance, the shape sensing is preferably in real-time. The processor 50 co-registers at least two of the three different data streams to a single reference frame. Where necessary, the imaging data 10, tracking data 20, and pose data 30 are rotated, translated and scaled—possibly both in the spatial and the temporal domain—to properly align the three data streams. The co-registration process will be illustrated in more detail further in this disclosure.

Finally, two or more of the co-registered data streams are simultaneously visualized on the display 60. Specific examples of simultaneously visualized data are given further in this disclosure.

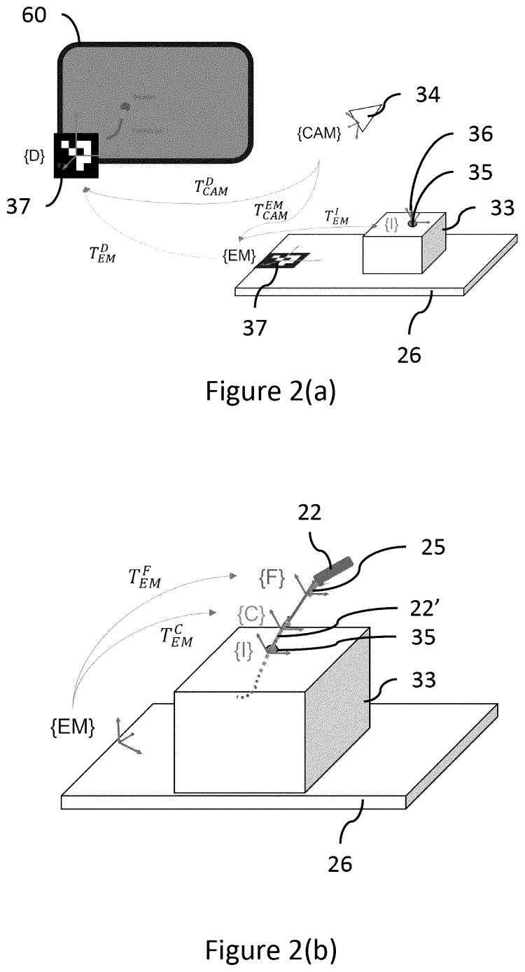

FIGS. 2(a) and 2(b) schematically illustrates an example of a simulated medical procedure. In this simulated medical procedure, when the instrument 22 is a (for instance virtual) fetoscope 22 and is inserted into a patient model 33. The patient model 33 takes the form of a simple cubic cavity in the embodiment of FIGS. 2(a) and 2(b). The fetoscope 22 is inserted into the patient model 33 through a cannula 22′ at the incision point 35. The fetoscope 22 and the incision point 35 are provided with respective electromagnetic (EM) trackers 25 and 36. The patient model 33 is placed on an EM field generator 26, which serves to track the position of both EM trackers 25 and 36.

In some embodiments, multiple reference frames exist: the incision point reference frame {I}, the cannula reference frame {C}, the fetoscope reference frame {F}, the EM field generator reference frame {EM} and the display reference frame {D}. In order to provide proper visual guidance, data originating in different reference frames needs to be co-registered. This requires knowledge of the transformation matrices between the different reference frames.

In order to calculate the transformation matrix

$${T}_{EM}^{D}$$

between the EM field generator 26 and the display 60, both the display 60 and the EM field generator 26 are correspondingly equipped with trackers or markers 37. A camera 34 is positioned such that the camera 34 can observe both markers; the camera 34 itself has a reference frame {CAM}. Since one of the markers 37 is fixedly attached to the EM field generator 26, the transformation matrix

$${T}_{EM}^{M}$$

between the EM field generator 26 and the marker 37 is known. By tracking both markers 37 with the camera 34, the transformation matrices

$${T}_{CAM}^{D} and {T}_{CAM}^{EM}$$

can be calculated. The transformation matrices

$${T}_{CAM}^{D} and {T}_{CAM}^{EM}$$

are the transforms from {CAM} to {D} and {EM} respectively. Then the transformation matrix between EM field generator reference frame {EM} and the display reference frame {D} can be obtained as

$${T}_{EM}^{D} = {( {T}_{CAM}^{EM} )}^{- 1} {T}_{CAM}^{D} .$$

Knowledge of the transformation matrix

$${T}_{CAM}^{D}$$

allows for the co-registration of data which originates from the EM field generator reference frame {EM} in the reference frame of the display 60. In this exemplary embodiment, data originating from the EM field generator reference frame {EM} may be the pose data of the patient model 33 fixedly attached to the generator 26. By continuous tracking of the markers 37, the transformation matrix is always known and calibration of the system is possible whenever the display 60 or the EM generator 26 are moved.

In the setup as shown, the data originating from the reference frames of the incision point, the cannula or the fetoscope may be first transformed to the reference frame of the EM field generator 26. The incision point EM tracker 36 provides the transformation matrix

$${T}_{EM}^{I}$$

and the fetoscope EM tracker 25 provides the transformation matrix

$${T}_{EM}^{F} .$$

The position and orientation of the cannula 22′ and the fetoscope 22 are visualized with respect to the incision point 35. When visualizing the 3D augmented reality (AR) content on the display 60, the incision point remains static and only the rotation and insertion of the cannula 22′ and the fetoscope 22 are represented. The rotations are preferably calculated in the display reference frame {D} to ensure that the AR instruments are always parallel with the real instruments. Since the fetoscope 22 is inserted through the cannula 22′, the two instruments are collinear, thus their rotations are the same. Therefore, the transformation matrix from the reference frames of the fetoscope and the cannula to the reference frame of the display can be expressed as

$${T}_{D}^{F} = {T}_{D}^{EM} {T}_{EM}^{F} .$$

The insertion length of the instruments is calculated as the distance between the calibrated incision point 35 and the points defined by the fetoscope EM tracker 25. In some embodiments, the incision point can be registered at the beginning of a surgery using an EM tracker, for instance, by registering a single point in the same {EM} frame. These translations are expressed in EM field generator reference frame {EM}. The obtained distances are the length of the instruments that are outside of the patient, l_{F,o} and I_{F,o}, for the fetoscope 22 and cannula 22′, respectively. By knowing the total length of these instruments, the insertion lengths are calculated as I_{F,i}=l_{F}−I_{F,o} and l_{C,i}=l_{C}−l_{C,o}, where l_{F} and l_{C} are the total length of the fetoscope 22 and the cannula 22′, respectively.

The fetoscope 22 consists of two parts, namely the rigid shaft and the flexible tip. The flexible tip consists of 10 cylinders, where each of the cylinders can rotate around the local z axis. This property is used to visualize the bending of the fetoscope. Bending of the fetoscope has a range of 90 degrees. This bending angle is determined through measurement of the physician's input. The virtual fetoscope 22 is assumed to behave according to a constant curvature model, where the rotation angle of each of the cylinders is equal and where the flexible tip can only bend in one plane. Under this assumption, the bending angle is then divided by the number of cylinders, and given as a rotation to each cylinder. Since each consecutive cylinder is a child of the previous cylinder, the resulting global bending angle of the 10th cylinder will be the same as the bending angle of the fetoscope: θₛ=θ_{F}/nₛ, where θₛ and θ_{F} are the rotation angle of each segment and the overall bending angle of the flexible tip, respectively, while nₛ is the number of segments that the flexible tip is composed of. The orientation of the plane in which the virtual fetoscope 22 can bend is given by the fetoscope EM sensor 25.

The obtained insertion length, orientation and bending angle (from which the shape is known) of the fetoscope 22 making up the tracking data, are transformed to the display reference frame. The real time 2D fetoscopic view—the image obtained by the tip of the fetoscope 22—is the imaging data in this embodiment. This imaging data is also transformed to the display reference frame and is set as the background of the visualization of the display. To generate the 3D view, two virtual cameras are placed in the scene for capturing the virtual content with a horizontal offset of 65 mm. The offset corresponds to the average interpupillary distance. The offset can be adjusted based on user preference. The two views from the cameras are stacked side-by-side generating the 3D image input for an autostereoscopic display. If the 2D fetoscopy image is to be visualized on a 3D display, such as for instance an autostereoscopic display or VR glasses, two additional virtual cameras are placed in the renderer. The image of one of these cameras will be visualized for the left eye of the user, the image of the other camera will be visualized for the right eye of the user. By setting both cameras to an identical position and orientation, an identical view is provided for the left eye and the right eye which results in the perception of a 2D image, even when using a 3D display. Alternatively, only one additional virtual camera can be placed in the scene and the image of this camera can be duplicated for the left and right eye of the user. Alternatively, the raw fetoscopy image may be visualized for both the left and right eye of the user. Furthermore, the visualization of the instrument can be positioned outside of the visualization of the imaging data or on top of the visualization of the imaging data, as the user can freely move it in the scene. This hybrid 2D and 3D visualization is preferably presented on an autostereoscopic display.

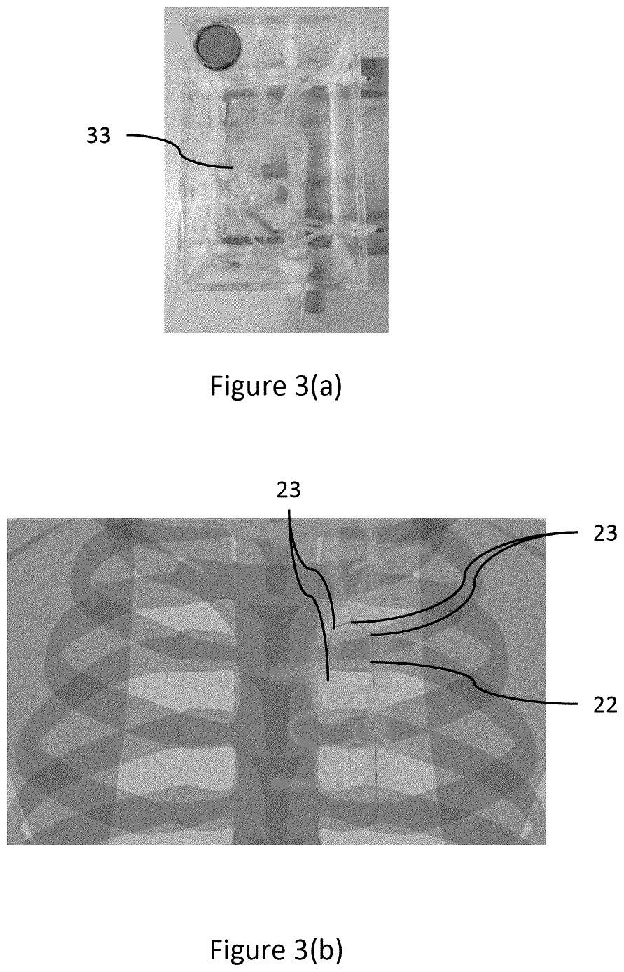

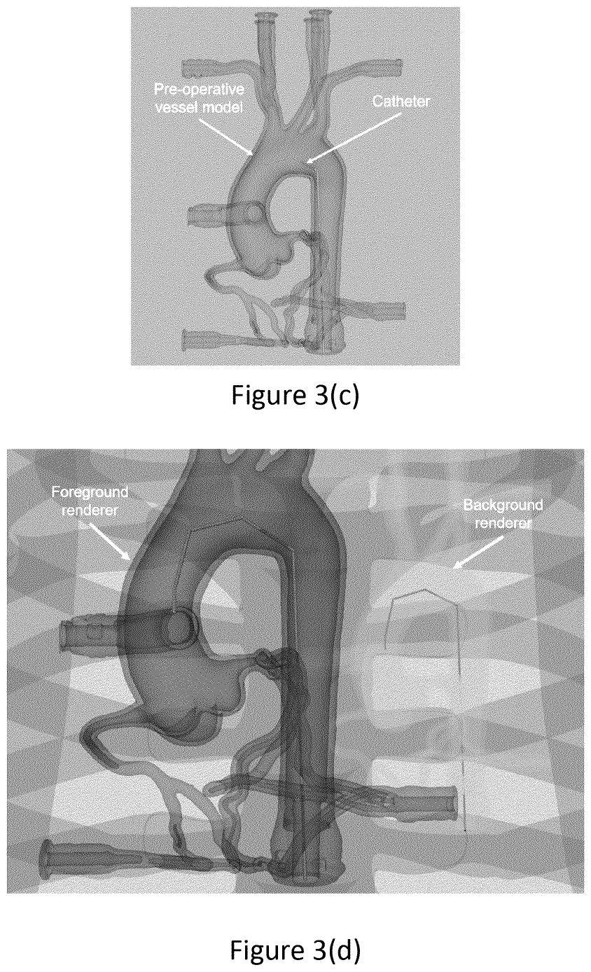

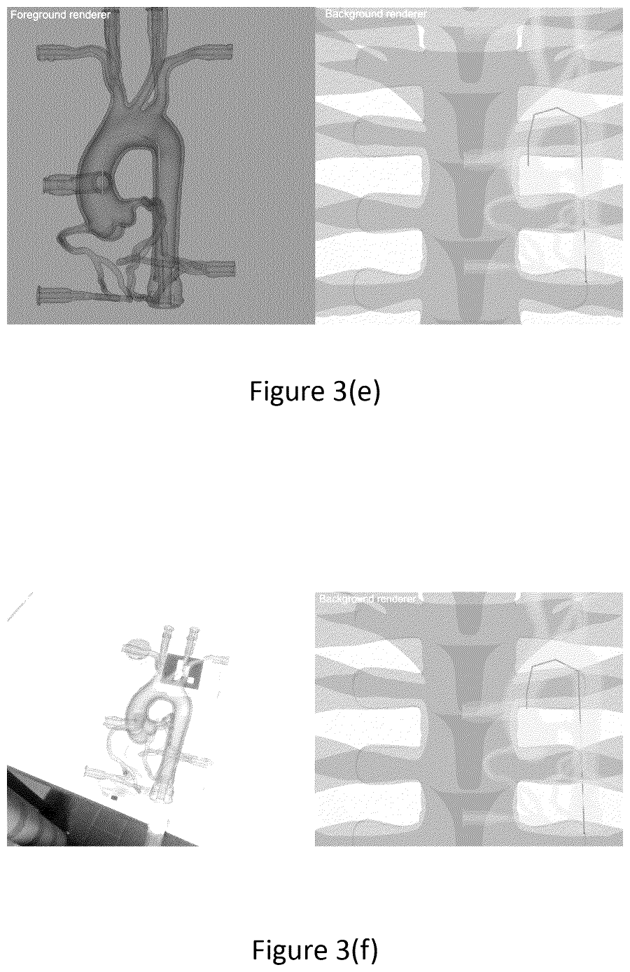

FIG. 3(a) shows a picture of a patient model 33. In the embodiment of FIG. 3(a), the patient model is a box trainer with a silicon coronary vessel model, employed for the simulation of cardiac catheterization. FIGS. 3(b)-3(f) all refer to the same embodiment.

In an exemplary embodiment where the instrument 22 is a catheter, the catheter 22 is inserted into the silicon vessel model. The catheter 22 comprises a distributed Fiber Bragg Grating (FBG) sensor that returns tracking data comprising the 3D positions of tracking points 23. The tracking data comprises at least three or more tracking points. Preferably, the spacing of the tracking points along the length of the catheter is adapted to the mechanical properties of the catheter such that every bending mode of the catheter can be tracked and visualized. For instance, the tracking data may comprise tracking points that are spaced apart along the length direction of the catheter. The distance between tracking points may vary according to the need of the instruments and the procedure. In some embodiments, the tracking points may be spaced apart by 1 mm. Preferably, the tracking points are not located along the centerline of the catheter but are distributed at a lateral distance of the centerline along two or more sides of the centerline. Such a distribution of tracking points ensures that three non-colinear tracking points are available at any time—also when the catheter is not bent. However, even when only a single fiber is located along the centerline of the catheter, the fiber will return non-colinear points once it is bent and colinear points when it is straight, making this arrangement useful as well for determining the position, orientation and shape of the catheter.

In some embodiments, the instrument being a flexible instrument is introduced into a target area of the medical procedure. The inserted portion of the flexible instrument is tracked in real time during the medical procedure to generate the tracking data. The tracking data includes tracking of the bending of the flexible instrument during the medical procedure.

Typically, intra-operative imaging data for a cardiac catheterization is delivered by a C-arm fluoroscope, which delivers 2D fluoroscopy images. For the purpose of a simulated catheterization, the fluoroscopy image can be simulated using the patient model 33 and a model of a ribcage. In FIG. 3(b), such a simulated fluoroscopy image is visualized. The imaging data and the tracking data of the catheter are co-registered and a visualization of the catheter 22 is added, based on the obtained 3D positions of the tracking points 23. The visualization of the catheter displays the position, orientation and shape of the catheter within the coronary vessels. Preferably, the view can be rotated along the x, y and z axes, thereby simulating the movements of a C-arm fluoroscope. In some embodiments, the first imager is configured to generate imaging data used to form real time image of the target region of the medical procedure.

If the 2D fluoroscopy image is to be visualized on a 3D display, such as for instance an autostereoscopic display or VR glasses, two virtual cameras are placed in the renderer. In some other embodiments, the 3D display may be a conventional stereoscopic display, an autostereoscopic display, a head-mounted display, binoculars of a stereomicroscope or a holographic display. The image of one of these virtual cameras will be visualized for the left eye of the user, the image of the other camera will be visualized for the right eye of the user. By setting both cameras to an identical position and orientation, an identical view is provided for the left eye and the right eye which results in the perception of a 2D image, even when using a 3D display. In this case the visualization of the catheter is also converted to a 3D visualization.

Through pre-operative imaging, model data of the coronary vessels can be obtained, for instance by means of CT 3D reconstruction. FIG. 3(c) shows a visualization of a 3D model of the patient model. Preferably, the color, transparency, orientation and scale of the visualization can be changed based on the preference of the user. The tracking data of the catheter is co-registered with the model data and visualized in the same scene. If the 3D model data and 3D tracking data are to be visualized on a 3D display, two virtual cameras are placed in the renderer. The image of one of these cameras will be visualized for the left eye of the user, the image of the other camera will be visualized for the right eye of the user. A translation is introduced between the positions of both cameras. Preferably, the translation can be adjusted based on the interpupillary distance (IPD) of the user. Preferably, the default translation between the positions of both virtual cameras is 65 mm, which corresponds to the mean IPD of humans. Since the contents of the left and right image are shifted with respect to one another, the user perceives a 3D image.

In some embodiments, the model data may be used to generate a 3D model image of the target region or target organ of the medical procedure. The processor of the system may be used to co-register the real time tracking data to pre-stored model data. During the medical procedure, the tracking data is used to provide 3D image rendering of the instrument corresponding to the 3D model image rendered using the model data. The 3D image is configured to provide placement of the instrument in the target region or the target organ during the medical procedure. The 3D image, in combination with the 2D image rendered simultaneously on the 3D display, may provide a more accurate location of the instrument during the medical procedure.

It is also possible to combine the two formerly mentioned visualization options in a single visualization. In FIG. 3(d), the background renderer comprises a 2D visualization of the 2D imaging data, co-registered with the 3D tracking data. The foreground renderer comprises a 3D visualization of the 3D model data, co-registered with the 3D tracking data. Alternatively, both visualizations can be displayed side-by-side, as shown in FIG. 3(e). This visualization mode is useful to avoid possible occlusions. Also in this visualization mode, the 3D model data can be rotated and scaled in accordance with user preferences or to align the 3D model data with the imaging data. Yet another visualization possibility is illustrated in FIG. 3(f). On the left, the foreground renderer comprises a visualization of the pose data of the patient model, which can be captured using a 2D or 3D camera. The tracking data is co-registered with the pose data and visualized on the same image. On the right, the background renderer comprises a 2D visualization of the 2D imaging data, co-registered with the tracking data. Preferably, the user can freely switch between the illustrated viewing modes.

According to an aspect of the disclosure, a system is disclosed for providing a visualization during a medical procedure on a patient or a simulated medical procedure on a patient model, the system comprising:
a. means for receiving imaging data of a first region of the patient's body or the patient model;
b. means for receiving tracking data of one or more instruments that are inserted in a second region of the patient's body or the patient model;
   the system being characterized in that it comprises
c. means for receiving model data of a fourth region of the patient's body or the patient model, and a processing means adapted for co-registering tracking data and model data; and
d. a display for simultaneous visualization of the co-registered data and the imaging data;
   and in that
c. the tracking data comprises three or more tracking points, wherein the three tracking points are not colinear; and/or one or more tracking points and a roll, pitch and yaw angle or a set of Euler angles or a set of quaternions or an angle-axis representation or a rotation matrix or a homogeneous transformation matrix; and/or a point cloud representation, for at least one of the one or more instruments;
d. the tracking data is used to determine a position and/or shape and/or orientation of at least one of the one or more instruments; and
e. the visualization comprises a representation of the shape and/or the position and/or the orientation of at least one of the one or more instruments.

Preferably, the system comprises:
f. means for receiving pose data of the patient's body or the patient model, the pose data belonging to a third region;
g. processing means for co-registering the imaging data, tracking data and pose data;
h. a display for simultaneous visualization of two or more of the first, second and third regions, and of the co-registered data belonging to the simultaneously visualized regions.

According to a further aspect of the disclosure, a method is disclosed for providing a visualization during a medical procedure on a patient or simulated medical procedure on a patient model, the method comprising the steps of:
a. receiving image data of a first region of the patient's body or the patient model;
b. receiving tracking data of one or more instruments that are inserted in a second region of the patient's body or the patient model;
the method being characterized in that it comprises
c. receiving model data of the patient's body or the patient model;
d. co-registering the tracking data and model data;
e. simultaneously visualizing imaging data and of the co-registered data;
   and in that
f. the tracking data comprises three or more tracking points, wherein the three tracking points are not colinear; and/or one or more tracking points and a roll, pitch and yaw angle or a set of Euler angles or a set of quaternions or an angle-axis representation or a rotation matrix or a homogeneous transformation matrix;
   and/or a point cloud representation, for at least one of the one or more instruments;
g. the tracking data is used to determine a position and/or shape and/or orientation of at least one of the one or more instruments; and
   the visualization comprises a representation of the shape and/or the position and/or the orientation of at least one of the one or more instruments.

In some embodiments of the method, the method further comprises the steps of:
a. receiving model data of a fourth region of the patient's body or the patient model;
b. co-registering the imaging data, tracking data and pose data;
c. simultaneously visualizing two or more of the first, second, third and fourth regions, and of the co-registered data belonging to the simultaneously visualized regions.

Reference signs

| | |
|---|---|
| imaging data | 10 |
| first region | 11 |
| imager | 12 |
| tracking data | 20 |
| second region | 21 |
| instruments | 22 |
| tracking points | 23 |
| instrument electromagnetic tracker | 25 |
| electromagnetic field generator | 26 |
| pose data | 30 |
| third region | 31 |
| patient's body | 32 |
| patient model | 33 |
| camera | 34 |
| incision point | 35 |
| incision point electromagnetic tracker | 36 |
| tracker or marker | 37 |
| processor | 50 |
| display | 60 |

1-34. (canceled)

35. A system for providing a visualization during a medical procedure on a patient or a simulated medical procedure on a patient model, the system comprising:
means for receiving imaging data of a first region of the patient's body or the patient model;
means for receiving tracking data of one or more instruments that are inserted in a second region of the patient's body or the patient model;
wherein
the system comprises means for receiving model data of a fourth region of the patient's body or the patient model, and a processing means adapted for co-registering tracking data and model data;
the system comprises a display for simultaneous visualization of the co-registered data and the imaging data;
the tracking data comprises three or more tracking points, wherein the three tracking points are not colinear; and/or the tracking data comprises one or more tracking points and/or a corresponding orientation by means of a corresponding roll, pitch and yaw angle, or a set of Euler angles or a set of quaternions, or an angle-axis representation or a rotation matrix, or a homogeneous transformation matrix, and/or a point cloud representation, for at least one of the one or more instruments;
the tracking data is used to determine a position and/or shape and/or orientation of at least one of the one or more instruments; and
the visualization comprises a representation of the position and/or the orientation of at least one of the one or more instruments, and a representation of a shape of at least one of the one or more instruments
wherein the tracking data is generated by one or more sensors that are distributed along and embedded in and/or fixedly attached to at least one of the one or more instruments, the distributed sensors being configured to conform to the shape of the instrument and to enable derivation of a shape of the instrument during the procedure.

36. The system according to claim 35, further comprising means for receiving pose data of the patient's body or the patient model, the pose data belonging to a third region, wherein the processing means are adapted for further co-registering pose data, wherein the display is adapted for further visualization of the pose data.

37. The system according to claim 35, wherein:
the model data and tracking data comprise 3D data;
a shape of the instruments is determined according to the tracking data comprising time-related data;
the display is a 3D display; and
a representation of the model data and the representation of a shape, position and/or orientation of the one or more instruments are 3D representations.

38. The system according to claim 35, wherein the imaging data is 2D data that is processed to allow for visualization on the 3D display.

39. The system according to claim 35, wherein there is at least a partial overlap between two or more regions and wherein the visualization comprises an overlay of the representation of the data originating from the two or more regions.

40. The system according to claim 39, wherein the overlay is presented aligned on corresponding data or wherein the overlay can be positioned and repositioned during the procedure such as to offer a desired view for a user.

41. The system according to claim 35, wherein a system latency is equal to or smaller than 50 ms.

42. The system according to claim 35, wherein the co-registration of the model data, imaging data, tracking data and orientation data occurs in a continuous or intermittent manner during the procedure.

43. The system according to claim 35, wherein the model data is generated prior to the procedure.

44. The system according to claim 35, wherein the imaging data is generated during the surgical procedure.

45. The system according to claim 35, wherein the imaging data is generated in an intermittent manner.

46. The system according to claim 35, wherein at least one of the distributed sensors for generating the tracking data comprises an optical fiber and/or at least one comprises one or more electromagnetic trackers.

47. The system according to claim 35, wherein the distributed sensors are configured to provide spatially resolved measurements along the length of the instrument, enabling derivation of the instrument shape in real time.

48. The system according to claim 35, wherein the distributed sensors are integrated such that the instrument can be used as an all-in-one device without the need for external tracking infrastructure.

49. A method for providing a visualization during a medical procedure on a patient or simulated medical procedure on a patient model, the method comprising the steps of:
receiving imaging data of a first region of a patient's body or the patient model;
receiving tracking data of one or more instruments that are inserted in a second region of the patient's body or the patient model;
receiving model data of a fourth region of the patient's body or the patient model;
co-registering the tracking data and model data;
simultaneously visualizing imaging data and the co-registered data; and
generating the tracking data by one or more sensors that are distributed along and embedded in or fixedly attached to at least one of the one or more instruments, the distributed sensors being configured to conform to the shape of the instrument and to enable derivation of a shape of the instrument during the procedure.

50. The method according to claim 49, further comprising receiving pose data of the patient's body or the patient model, the pose data belonging to a third region, further co-registering pose data, and further visualizing of the pose data.

51. The method according to claim 49, wherein:
the model data and tracking data comprise 3D data;
the display is a 3D display; and
a representation of the model data and the representation of a shape, position and/or orientation of the one or more instruments are 3D representations.

52. The method according to claim 49, wherein the imaging data is 2D data that is processed to allow for visualization on the 3D display.

53. The method according to claim 49, wherein there is at least a partial overlap between two or more regions and wherein the visualization comprises an overlay of the representation of the data originating from the two or more regions.

54. The method according to claim 53, wherein the overlay is presented aligned on corresponding data or wherein the overlay can be positioned and repositioned during the procedure such as to offer a desired view for a user.
